(19) United States
(12) Patent Application Publication
(10) Pub. No.: US 2018/0201426 A1
(43) Pub. Date: Jul. 19, 2018

(54) Intracanalicular Plugs with Improved Absorption Period and Other Identifications

(71) Applicant: Paragon BioTeck, Inc., Portland, OR (US)

(72) Inventors: Patrick H. Witham, Eugene, OR (US); Sailaja Machiraju, Beaverton, OR (US); Lauren Mackensie-Clark Bluett, Scappoose, OR (US)

(21) Appl. No.: 15/407,264

(22) Filed: Jan. 17, 2017

(51) Int. Cl. B65D 77/26; A61F 9/007; A61F 9/00; B65D 79/00; A61B 50/30

(52) U.S. Cl. CPC B65D 77/26; A61F 9/00772; A61F 9/0017; A61F 2250/0087; A61B 50/30; A61F 2250/0067; B65D 79/00

(57) ABSTRACT

A system for identifying intracanalicular plugs, wherein the plugs are packaged in holders in a box which contains the plugs optionally packaged in a pouch is provided, whereby identification and tracking of hard-to-identify plugs are facilitated. The package, pouch, holders are marked with plug characteristic indicators such as absorption time and drug substance contained therein, brand indicators and other characteristics of the plugs. An array of such identifiably packaged plugs with different characteristics are also provided.

### FIELD OF THE INVENTION

The present invention relates generally to packaging for intracanalicular punctal occluders (plugs) and systems relating thereto, and, more particularly, systems for facilitating selection and identification of punctal plugs by a health care professional.

### BACKGROUND OF THE INVENTION

Intracanalicular punctal occluders, referred to herein as plugs, are conventionally utilized to block tear drainage by the occlusion of the canaliculus. Such intracanalicular plugs are frequently formed of bio-absorbable or degradable materials. Absorbable intracanalicular plugs are intended to temporarily block tear drainage by occlusion of the canaliculus in order to determine the potential effectiveness of permanent occlusion, to temporarily treat dry eye syndrome and the dry eye components of various ocular surface diseases, to temporarily enhance the efficacy of topical medications or ocular lubricants, to temporarily treat contact lens intolerance secondary to dry eye, and/or to temporarily treat dry eye after ocular surgery.

Intracanalicular plugs are usually supplied to medical practitioners mounted on holders made of foam or similar materials that have spaced grooves, wherein the intracanalicular plugs are held. The plugs can be removed for example utilizing a device similar to a jeweler's type forceps or other similarly sized instruments.

Typically, the holder has a shape defining two grooves, each groove receiving a single intracanalicular plug. In use, the tip of an elongated instrument is inserted in the hollow center of an intracanalicular plug while the intracanalicular plug is in the holder to remove the intracanalicular plug, and the instrument can be used to place the intracanalicular plug in the patient's lacrimal punctum. Many intracanalicular plug and inserter designs are available in the art.

Generally, intracanalicular plugs are made of absorbable material such that the intracanalicular plug will be absorbed into the body over a predetermined period of time after its placement in the body. Depending on the intended use and desired duration of occlusion, the material used for the intracanalicular plugs differ. These punctal plugs may also be embedded with one or more suitable pharmaceutical substances, such as an antimicrobial agent. The punctal plugs may sometimes also act as medicine carriers or aid in release of the drug, or for administering an active agent, into the tissues in and around one or both eyes of the subject.

U.S. Pat. No. 8,591,484 to Gubachy et al. discloses methods and apparatus for treatment of dry eyes by providing a single apparatus for measuring the diameter of the lacrimal punctum and, based on the measured punctal diameter, inserting therein an appropriately sized punctum plug.

U.S. Patent Publication No. 2004/0068286 discloses a punctum dilating and plug inserting system which includes an inserter with a longitudinal body and a protective cover defining a dilator. A packaging is also provided for receiving, protecting, and handling at least a portion of the entire inserter until ready for use.

Despite the large number of absorbable intracanalicular plugs available, they all have confusingly similar appearances to the naked eye. They all have very similar sizes, shapes and color, and due to the nature of the materials used and the fact that they must be implanted, are not amenable to labeling or marking for differentiation. In practice, without the packaging of the plugs, a healthcare professional, e.g. a surgeon, or his or her assistant, will not be able to discern the differences between various plugs.

U.S. Patent Publication No., 2004/0068235 to Hallam discloses a packaging system for a punctum plug insertion device having a punctum plug loaded thereon, including a clear plastic tube containing the device and a sterile barrier end cap enclosing an open end of the tube. The end cap may be provided with visual indicia to indicate a size of punctum plugs loaded on the insertion device. In addition, the device may be color coded to indicate a size of the plug loaded on the insertion device. A carton is also provided for storing the tubes. Each of the instruments may be molded in plastics of various colors or otherwise color coded, with each color associated with a single size of punctum plug which is mounted on the instrument. As such, a quick visual inspection of the instrument provides identification of the size of the plug thereon.

Therefore there is a need for a system that solves the above problems of the prior art.

### SUMMARY OF THE INVENTION

The present invention overcomes the disadvantages of the prior art by utilizing a color coding system to assist a practitioner to easily identify particular intracanalicular plugs and prevent inadvertent use of plugs with incorrect absorption times or adverse events associated with specific materials, such as allergic reactions.

In accordance with one embodiment of the present invention, to facilitate the recognition of particular intracanalicular plugs by a practitioner, an article is provided which comprises: a) one or more intracanalicular plugs mounted on a holder, wherein the holder is enclosed in a package; and b) a first plug characteristic indicator disposed on the package, the first plug characteristic indicator including i) an absorption time identifier for indicating the absorption time of the intracanalicular plug mounted on the holder, and c) a second plug characteristic indicator disposed on the holder, wherein the second plug characteristic indicator is substantially the same as the first plug characteristic indicator, wherein the first or second plug characteristic indicator allows a user of the plug to identify the plug for its appropriate intended usage.

In accordance with one embodiment, the article of the invention further comprises a brand indicator disposed on the package.

In accordance with one embodiment, the article of the invention further comprises a brand indicator disposed on the holder.

In accordance with one embodiment, in the article of the invention, the first or second plug characteristic indicator may further comprise a drug substance indicator for indicating one or more pharmaceutical substance contained in or on the plug.

In accordance with one embodiment, the first or second plug characteristic indicator may include a common color scheme, which may indicate the source or manufacturer of the plug.

In accordance with one embodiment, the article of the invention further comprises one or more instructional indicia which associate the common color scheme with one or more specific characteristics of the intracanalicular plug. In one embodiment, the instructional indicia is disposed on the package or on an item such as a piece of paper as an insert enclosed in the package. In one embodiment, the indicators could be visual indicia (for example a color scheme or pictorial representations) or narrative indicia (e.g. an alphanumerical symbol).

In one embodiment, the article of the present invention is marked and sold under the trademark of PARAGON. The absorption periods of the plugs may range from a few days to a few months. In one embodiment, the plugs contained in the article of the present invention have an absorption period of 180 days. In another embodiment, the absorption period is 90 days.

In accordance with one embodiment, the present invention provides an array of articles, comprising at least a first article and a second article, wherein the first article comprises one or more first intracanalicular plugs mounted on a first holder, wherein the first holder is enclosed in a first package; and a first plug characteristic indicator disposed on the first package, the first plug characteristic indicator including a first absorption time identifier for indicating the absorption period of the first intracanalicular plug mounted on the holder, and a second plug characteristic indicator disposed on the first holder, wherein the second plug characteristic indicator is substantially the same as the first plug characteristic indicator, wherein the first or second plug characteristic indicator allows a user of the plug to identify the first intracanalicular plug for its appropriate intended usage; the second article comprises one or more second intracanalicular plugs mounted on a second holder, wherein the second intracanalicular plugs is different from the first intracanalicular plugs, and the second holder is enclosed in a second package; and a third plug characteristic indicator disposed on the second package, the third plug characteristic indicator including a second absorption time identifier for indicating the absorption period of the second intracanalicular plug mounted on the holder, and a fourth plug characteristic indicator disposed on the second holder, wherein the fourth plug characteristic indicator is substantially the same as the third plug characteristic indicator, and wherein the first and third plug characteristic indicators are visually distinct from each other, wherein the third or fourth plug characteristic indicator allows a user of the plug to identify the second intracanalicular plug for its appropriate intended usage; and an outer container enclosing the first package and the second package, wherein the outer container includes a container indicia which includes both the first and third plug characteristic indicia.

In another embodiment, the first, third, and container indicia all comprising a common color scheme.

In another embodiment, the array of articles according to the present invention may further comprise an instructional indicia explaining association between one indicia (e.g. a color, a logo, a symbol) with a characteristic (e.g. the absorption period, the drug substance embedded therein, or size) of the plugs, and other information.

Other aspects and advantages of the present invention will become apparent from the following description of the invention taken in conjunction with the accompanying drawings wherein like parts in each figure are identified by the same reference character

### BRIEF DESCRIPTION OF THE DRAWINGS

FIG. 1 shows a box for containing intracanalicular plugs and holders in accordance with the present invention.

FIG. 2 is a view of an intracanalicular plug holder holding a pair of intracanalicular plugs for use with the present invention.

FIG. 3 is a side view of the holder shown in FIG. 2.

FIG. 4 is a sectional view of an exemplary intracanalicular plug for use with the present invention.

FIG. 5 is a perspective exploded view showing boxes for use in a color coding system in accordance with the present invention.

FIG. 6 shows a pouch for packaging holders of the present invention.

FIG. 7 is a view of an intracanalicular plug holder holding a pair of intracanalicular plugs for use with the present invention, having a different color and shape than that shown in FIG. 2.

### DETAILED DESCRIPTION OF THE INVENTION

The present invention provides a systematic coding system for the packaging and labeling of punctal plugs, especially absorbable plug, which clearly classify and differentiate a range of plugs based on their duration of occlusion, intended use in the anatomy of the eye, or material used to produce the punctal plug. The coding system eliminates confusion arising from two or more punctal plugs which look alike or are otherwise difficult to differentiate to the naked eye.

Punctal plugs have been used for more than 20 years for the symptomatic relief of Dry Eye Disease (DED). Punctal plugs are tiny, biocompatible devices inserted into tear ducts to block tear drainage. The occlusion process is intended to increase the volume of the eye's naturally produced tear film and surface moisture in an effort to reduce symptoms commonly associated with DED. Punctal plugs occlude the nasolacrimal duct, thereby reducing tear drainage through the upper and/or lower punctum in the eye. These devices often are no larger than a grain of rice.

Based on the point of insertion, punctal plugs are grouped into 2 groups: (1). Punctal (or punctum) plugs, which are placed at the top of the puncta. The tops of these plugs are often visible to the patient looking carefully in a mirror. Generally speaking, the advantage of punctal plugs is ease of removal, with the accompanying disadvantage that they may more easily be lost. (2). Intracanalicular plugs, which are inserted into the canalicula. Most plugs of this type cannot be seen after insertion. They cannot be removed in the way punctal plugs can, though they may be flushed out with irrigation.

In practice often all types of plugs collectively are referred to as punctal plugs. Some of these are inserted into the punctum as a liquid and then they harden and conform to the individual's drainage system. Others start out rigid and become soft and flexible, adapting to the individual's punctal size after they are inserted.

When manufactured, intracanalicular absorbable punctal plugs are generally mounted on a carrier or holder, sealed in a foil pouch or other packaging material that acts as a moisture barrier, and then packaged in a secondary Tyvek® pouch which is finally packed into the outer box. In accordance with one embodiment of the present invention, the labeling materials of the package (that is, most or all of the foil pouch, secondary pouch and the outer box) and the inner most carrier or holder are coded (e.g. via the use of a color scheme) in a similar systematic fashion to reflect the intended use of the plug.

Currently, due to the manufacturing process and the need to be subjected to sterilization, the material used to mount the plug in the present invention is a foam carrier or holder, which is not amenable for labeling by printing or other method of labeling other than color or shape. In one embodiment of the present invention, the holders are coded via color and/shape of the holder consistently with the outer packaging (pouches and box) and the labeling information thereon.

Since the foam carrier or holder is not discarded until the time the plug is inserted into the patient's anatomy, the coding system of the present invention reduces the risk of the plugs getting mixed up at the physician's office by providing a clear differentiation between available plug types, mitigates or eliminates the risk of incorrect plugs getting implanted.

The coding system of the present invention is applicable for absorbable or non-absorbable punctal plugs based on the material used to produce them. The material used to produce these punctal plugs may be but not limited to collagen, synthetic biocompatible hydrophilic polymers (e.g.: polymers (Polycaprolactone (PCL), Polydioxanone (PDO), Poly (Glycolide-co-caprolactone) (PGCL), etc.), hydrogels (e.g.: hydroxyethylmethacrylate (HEMA), methyl methacrylate), polytetrafluorethylene (known by the trademark TEFLON), or silicon. Depending on the material used for an absorbable punctal plug, the duration of occlusion ranges from a few days to several months. Non-absorbable plugs, used for long-term occlusion are most commonly produced of silicone or a hydrophilic acrylic.

Temporary or dissolvable punctal plugs usually last from a few days to as long as several months. These types of plugs may be used but not limited to circumstances such as preventing dry eyes after LASIK. Dissolvable, temporary punctal plugs sometimes are used to determine if the treatment works for the dry eye condition. If so, then semi-permanent punctal plugs might be considered. These punctal plugs can also be used as a treatment option for seasonal allergy patients experiencing dry eye symptoms. Punctal plugs usually are considered when non-prescription or prescription eye drops fail to relieve your dry eye condition.

In some cases these punctal plugs may be embedded with an antibacterial agent like irgacare MP, chlorohexidine or any other antimicrobial agent that's compatible with the invention. These punctal plugs may sometimes act as medicine carriers or aid in release of the drug, or to administering an active agent, i.e. a medicine or medication, to a subject by transdermal or other surface absorption of the agent into the tissues in and around one or both eyes of the subject.

These punctal plugs may be dyed or undyed and if dyed, FDA approved color additive for medical devices is used (eg: FD & C Violet #2). These devices may be available in 2 types of packages options, sterile preloaded and non-sterile bulk packs. They may or may not be available accompanied with an insertion tool, which may or may not be loaded with the punctal plugs.

In general the size of these punctal plugs ranges from 0.2 mm diameter to over 1 mm but the majority of plugs are available in the 0.3 to 0.5 or 0.6 range.

These punctal plugs have many designs and shapes, including but not limited to; umbrella (which do not “disappear” into the tear duct, making them easy to spot and remove if necessary), tapered (this design exerts extra force horizontally to help keep the punctal plug in its proper place), cylindrical, hollow (a hollowed interior can help the punctal plug adhere to the shape of the eye's tear duct), reservoir (this style captures and holds tears, which helps reduce foreign body sensation and increase comfort), or slanted or low profile cap (this design can help maintain comfort, while providing extra stability).

In one embodiment, suitable punctal plugs include those for insertion through the punctal opening into the canaliculus in order to block tear drainage through the lacrimal drainage system. They are made from an absorbable materials, and are available in various sizes to accommodate different patient physiologies and to achieve occlusion of the canaliculus (or punctum). The polydioxanone punctal plugs are 1.75 mm long and are available in multiple sizes ranging from 0.3 mm to 0.5 mm in diameter and the poly (glycolide-co-caprolactone) punctal plugs are 1.75 mm long and are available in multiple sizes ranging from 0.3 mm to 0.5 mm in diameter.

Both the plugs are packaged in similar fashion differing only in the color of their packaging and labeling material. In one example, two plugs are included in each package, so that a plug can be placed in each canaliculus. There may be ten packages in each box and can be used in case a plug is dropped or mishandled or for multiple use.

The polydioxanone plugs have a degradation rate of approximately 180 days while the poly (glycolide-co-caprolactone) have a degradation of 90 days. These plugs can mainly be used in the cases where short-term occlusion is recommended. These plugs are used to determine the potential effectiveness of permanent occlusion, temporarily treat dry eye syndrome, and the dry eye components of various ocular surface diseases. These plugs can also be used for temporarily enhancing the efficacy of topical medications or ocular lubricants, temporarily treat contact lens intolerance secondary to dry eye, and temporarily treat dry eye after ocular surgery.

The material used for packaging of punctal plugs may be but not limited to cardboard, paper, plastic, foam or any type of synthetic material used for medical device packaging.

Now referring to the figures, several specific examples of the present invention are described.

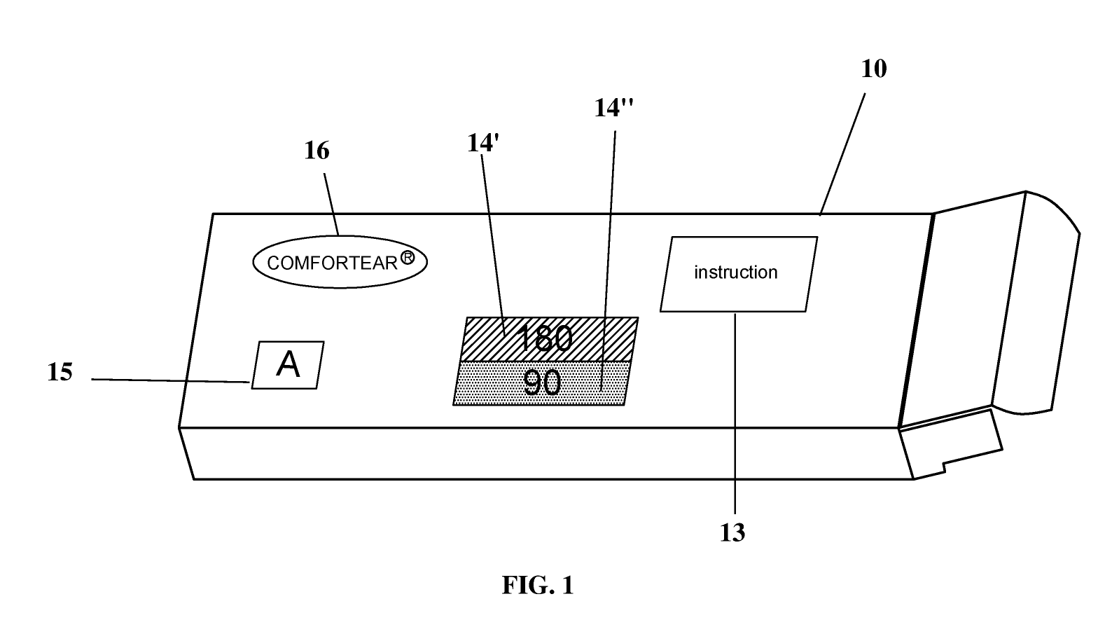

FIG. 1A shows a box in accordance with the present invention, wherein the box having one or more portions on the exterior marked by numerical figures identifying various characteristics of the plugs, such as the intended duration of the intracanalicular plugs carried therein. Additional indicia could also be included to describe various coatings or other attributes not visible to the naked eye.

The intracanalicular plugs are each mounted on a holder that is color coded consistent with the packaging for the specification from which the intracanalicular plug is made or other characteristics of the intracanalicular plugs like duration of plug. Since the holder will not be discarded until the intracanalicular plugs are inserted in the body, the risk factor associated with misidentifying intracanalicular plugs is obviated.

More specifically, in accordance with the present invention, intracanalicular plugs can be color coded differently by mounting them on differently colored holders and matching the intracanalicular plugs by putting a symbol of the same color on the associated box or carton label, or package insert.

A plurality of intracanalicular plugs mounted on one or more holders are packaged in a box. The intracanalicular plugs are made of an absorbable material having a predetermined duration of absorption in the canaliculus of the eye, and each absorption time period is identified by a distinct color of the holder, the same color being indicated on the box.

As indicated above, absorbable materials typically used to manufacture the intrananalicular plugs include polydioxanone (PDO), Polycaprolactone (PCL), Poly (Glycolide-co-caprolactone) (PGCL). Polyglycolic Acid (PGA), copolymer of polyglycolic acid and trimethylene carbonate, and various types of collagen. The absorption period ranges from few days to up to few months. An example of such type of intracanalicular punctal plug is a Polydioxanone plug which is used for occlusion therapy up to 180 days.

The plugs may be embedded with one or more suitable pharmaceutical compositions such as an antimicrobial agent, e.g. an antibacterial agent like irgacare MP, chlorohexidine.

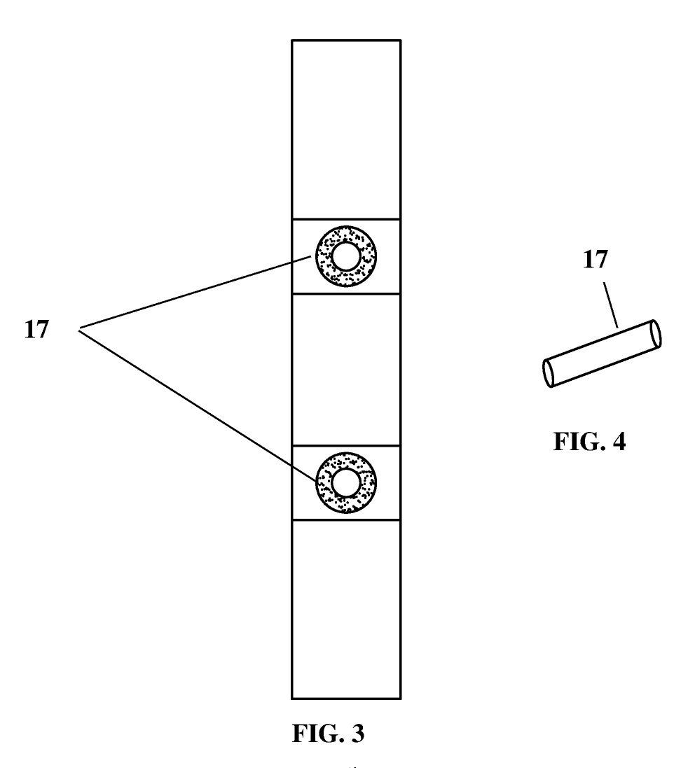

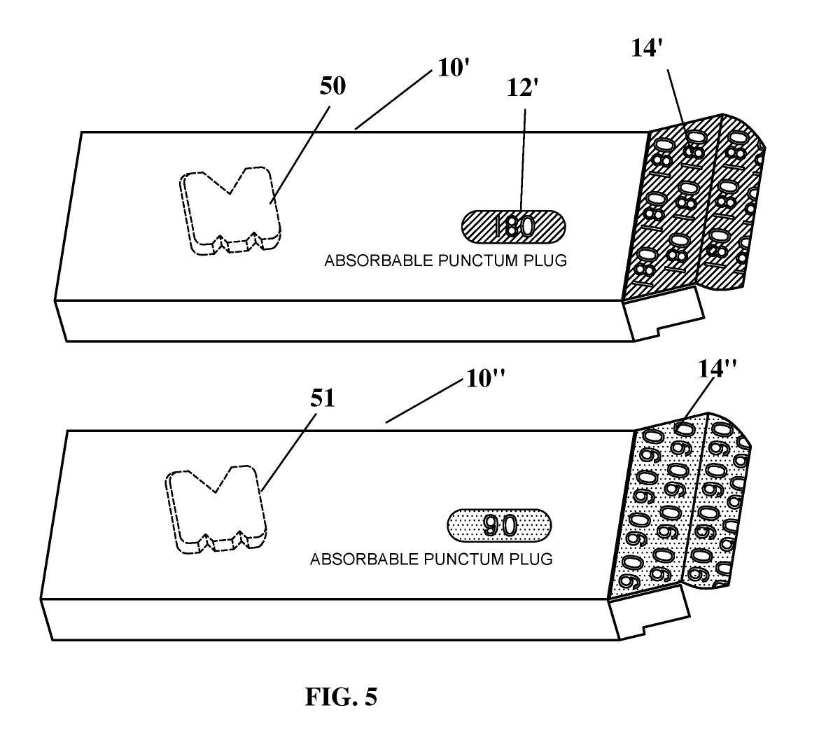

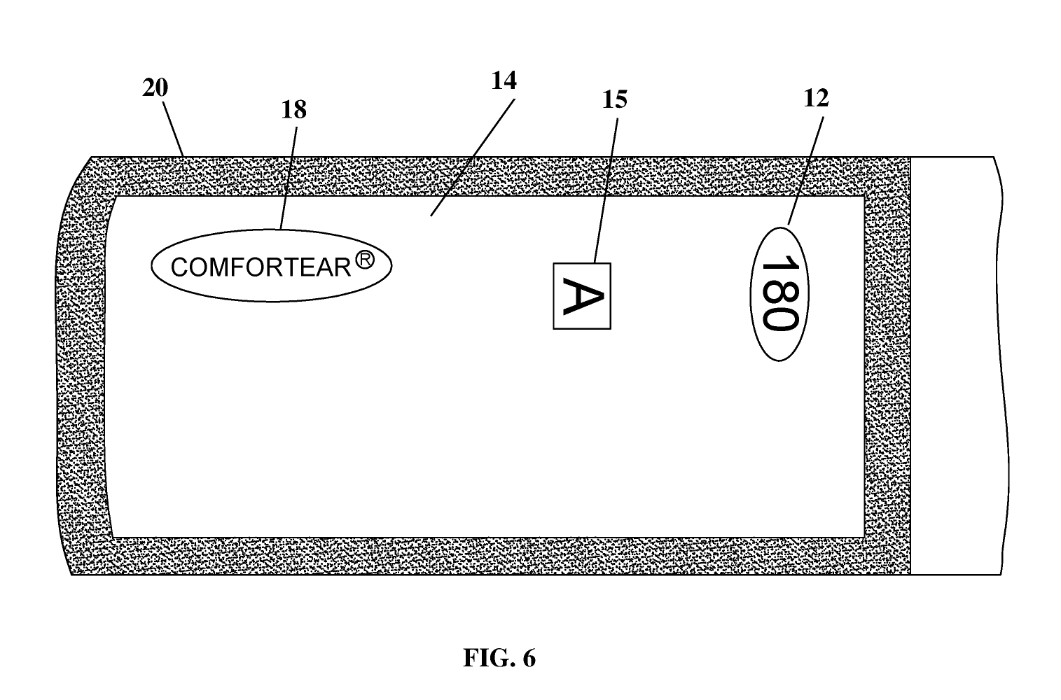

FIG. 1 and FIG. 5 together show a box or carton 10 for use with the present invention, the box being of conventional construction with the exception that portions 12 and 14 of the box display a distinct numerical figure identifying the intended duration or other characteristic of the intracanalicular plugs and the color of the holders therein. For example, as shown in FIG. 1, the number “180” can be seen on the front panel of the box at 12 and at the end of the box at 14 designed to fold along the front panel to form a space to receive a plurality of intracanalicular plugs 17 held in holders 50, as shown in FIGS. 3-4, which may be packaged into a pouch shown in FIG. 6 The intracanalicular plugs mounted on holders are enclosed in the box 10 with the intracanalicular plugs held in spaced grooves formed in the holders as shown at 17 and 18. The holders are typically made of foam or other soft, semi-rigid materials to be light in weight but to securely hold the plugs. The holders are of a distinct color 14′ or 14″ associated with the intracanalicular plugs to be held thereby.

The intracanalicular plug foam holder normally has open ends accessible by an instrument which is also used to implant the intracanalicular plugs in the lacrimal punctum of the eye. The distinct color of the holder 14 corresponds with the distinct color 14′ or 14″ on the box to facilitate identification of the particular intracanalicular plugs supplied therein. When the color coding of the present invention is utilized as part of a color coding system, it should be appreciated that a plurality of holders can be supplied in a single box or multiple boxes even though a single box is often used for each type of intracanalicular plug and holder therefor.

On the box 10, additional indicia are used to indicate other characteristics of the plugs, such as drug material indicator 15 identifying the medicinal substances or drugs embedded in the plugs; and an appropriate trademark 16. The plug absorption time 12′ can be separately indicated from the color block 14′ & 14″, if the box contains plug holders having more than one color, or the number can be printed on the color block, as shown on the end panel, if the box contains holders having only one color.

FIG. 6 shows a pouch 20 which contains one or more holders. The pouch may be sealed airtight, and made of foil or plastic materials (not shown). The pouch may contain some or all of the indicia on the box 10.

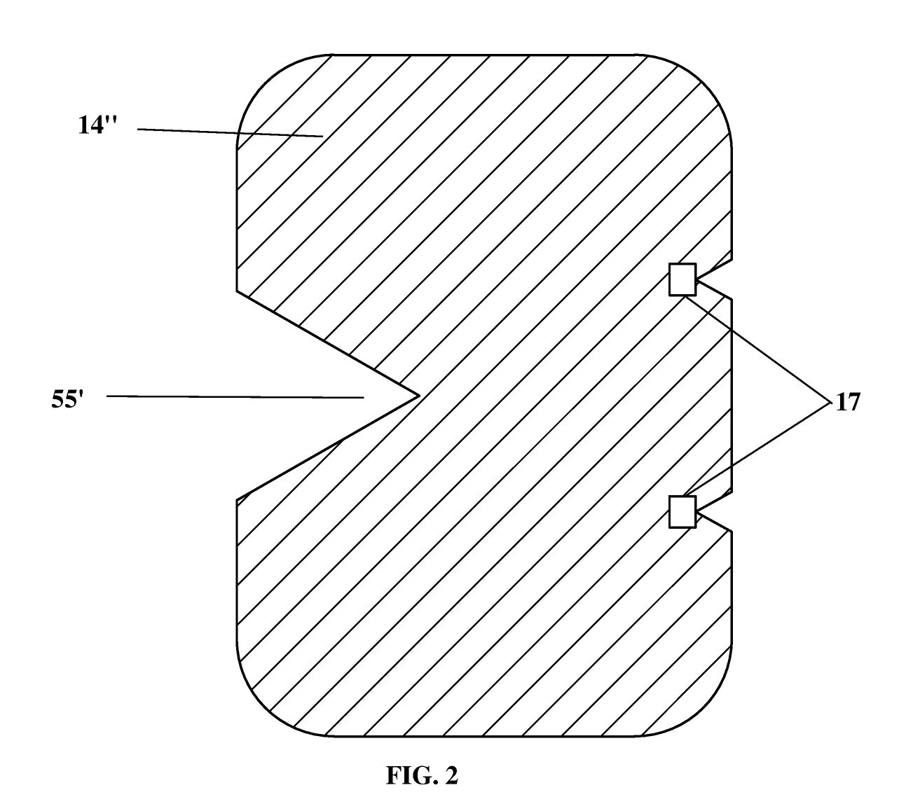

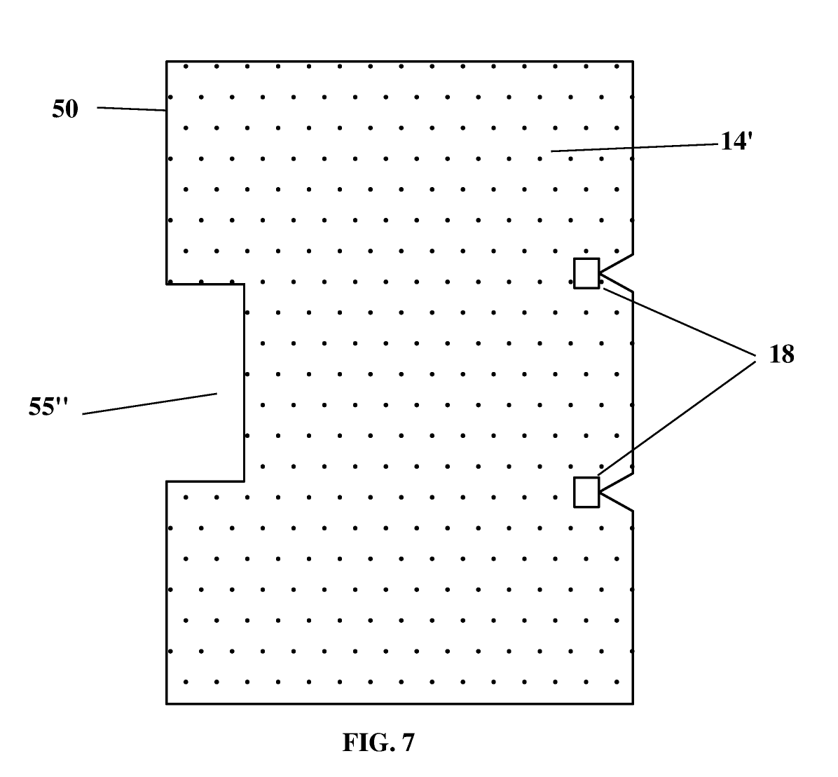

FIG. 7 shows a foam holder having a different shape than that shown in FIG. 2.

The package, according to the present invention, is utilized to supply intracanalicular plugs to the practitioner in a manner to facilitate identification of the particular plugs being delivered. The color coding system of the present invention can be utilized to supply intracanalicular plugs to a practitioner in a manner to facilitate identification of the particular plugs. In most cases, the intracanalicular plugs will be identified relative to the nature of the materials related to their absorption rates from which the intracanalicular plugs are made. However, the system of the present invention can be utilized to identify any particular feature of intracanalicular plugs.

A color coding system for intracanalicular plugs in accordance with the present invention is shown in FIG. 5 wherein boxes 10′ and 10″ are utilized to supply and/or deliver holders and intracanalicular plugs 50 and 51, respectively, that could be used by a practitioner for a procedure on a single patient or on multiple patients. Box 10′ has sections 12′ and 14′ displaying the same distinct color as the holder 50 along with a symbol, such as “180” identifying the particular absorbable material from which the intracanalicular plug is made. Similarly, box 10″ has sections 12″ and 14″ displaying a distinctly different color from the color displayed on box 10′ such that the sections, 12″ and 14″ displaying the holder 51 along with a symbol such as “90” identifying the particular absorbable material from which the intracanalicular plug is made. Accordingly, the boxes can be presented at the same time to a practitioner to provide a color coding system for the intracanalicular plugs.

In as much as the present invention is subject to many variations, modifications and changes in detail, it is intended that all subject matter discussed above or shown in the accompanying drawings be interpreted as illustrative only and not be taken in a limiting sense.

The invention has been illustrated by the above descriptions and examples. The examples are not intended to be limiting in any way. As used throughout, ranges are used as shorthand for describing each and every value that is within the range. Any value within the range can be selected as the terminus of the range. In addition, all references cited herein are hereby incorporated by reference in their entireties.

1. An article comprising:
a) one or more intracanalicular plugs mounted on a holder, wherein the holder is enclosed in a package; and
b) a first plug characteristic indicator disposed on the package and in the form of a physical attribute selected from the group consisting of colors and shapes, the plug characteristic indicator including
i) an absorption time identifier for indicating the absorption period of the intracanalicular plug mounted on the holder,
and
c) a second plug characteristic indicator disposed on the holder, wherein the second plug characteristic indicator is substantially the same as the first plug characteristic indicator,
wherein the first or second plug characteristic indicator allows a user of the plug to identify the plug for its appropriate intended usage.

2. The article of claim 1, further comprising a brand indicator disposed on the package.

3. The article of claim 2, further comprising a brand indicator disposed on the holder.

4. The article of claim 1, wherein the first or second plug characteristic indicator further comprises
ii) a drug substance indicator for indicating one or more pharmaceutical substance contained in or on the plug.

5. The article of claim 1, wherein the first or second plug characteristic indicator includes a common color scheme.

6. The article of claim 5, wherein the color scheme comprises the same color or the same pattern of colors.

7. The article of claim 1, further comprising one or more instructional indicia which associate the plug characteristic indicators with one or more specific characteristics of the intracanalicular plug.

8. The article of claim 7, wherein the instructional indicia is disposed on the package or an insert enclosed in the package

9. The article of claim 1, wherein the holder is contained in a pouch which comprises the same plug characteristic indicators as disposed on the package.

10. The article of claim 9, wherein the pouch further comprises a brand name indicator.

11. An array of articles, comprising at least a first article and a second article, wherein
A) the first article comprises
a) one or more first intracanalicular plugs mounted on a first holder, wherein the first holder is enclosed in a first package; and
b) a first plug characteristic indicator disposed on the first package, the first plug characteristic indicator including
i) a first absorption time identifier for indicating the absorption period of the first intracanalicular plug mounted on the holder,
and
c) a second plug characteristic indicator disposed on the first holder, wherein the second plug characteristic indicator is substantially the same as the first plug characteristic indicator,
wherein the first or second plug characteristic indicator allows a user of the plug to identify the first intracanalicular plug for its appropriate intended usage;
B) the second article comprises
a) one or more second intracanalicular plugs mounted on a second holder, wherein the second intracanalicular plugs is different from the first intracanalicular plugs, and the second holder is enclosed in a second package; and
b) a third plug characteristic indicator disposed on the second package, the third plug characteristic indicator including
i) a second absorption time identifier for indicating the absorption period of the second intracanalicular plug mounted on the holder,
and
c) a fourth plug characteristic indicator disposed on the second holder, wherein the fourth plug characteristic indicator is substantially the same as the third plug characteristic indicator, and wherein the first and third plug characteristic indicators are visually distinct from each other,
wherein the third or fourth plug characteristic indicator allows a user of the plug to identify the second intracanalicular plug for its appropriate intended usage; and
C) an outer container enclosing the first package and the second package, wherein the outer container includes a container indicia which includes both the first and third plug characteristic indicia.

12. The array of articles of claim 11, wherein the first, and third indicia comprise a first color scheme.

13. The array of articles of claim 12, wherein the second, and fourth indicia comprise a second color scheme distinct from the first color scheme.

14. The array of articles of claim 11, further comprising a brand indicator disposed on the package.

15. The array of articles of claim 14, further comprising a brand indicator disposed on the holder.

16. The array of articles of claim 11, wherein the first or second plug characteristic indicator further comprises
ii) a drug substance indicator for indicating one or more pharmaceutical substance contained in or on the plug.

17. The array of articles of claim 11, further comprising one or more instructional indicia which associate the plug characteristic indicators with one or more specific characteristics of the intracanalicular plug.

18. The array of articles of claim 17, wherein the instructional indicia is disposed on the package or an insert enclosed in the package.

19. The array of articles of claim 11, wherein the holder is contained in a pouch which comprises the same plug characteristic indicators as disposed on the package.

20. The array of articles of claim 11, wherein the package further comprises a brand name indicator.

21. The array of articles of claim 11, wherein the package comprises the first and second plug characteristics, the brand indicator, and the first and second color schemes.
